(19) United States
(12) Patent Application Publication
(10) Pub. No.: US 2025/0002521 A1
(43) Pub. Date: Jan. 2, 2025

(54) Method for purifying sucralose-6-ester

(71) Applicant: ANHUI JINHE INDUSTRIAL CO., LTD., Chuzhou City (CN)

(72) Inventors: Zhengsong ZHANG, Chuzhou City (CN); Dongdong SHEN, Chuzhou City (CN); Hao CI, Chuzhou City (CN); Xin QIU, Chuzhou City (CN)

(21) Appl. No.: 18/709,585

(22) PCT Filed: Jul. 7, 2021
(86) PCT No.: PCT/CN2021/104937

§ 371 Date: May 13, 2024

(51) Int. Cl. C07H 13/04; C07H 1/06

(52) U.S. Cl. CPC C07H 13/04; C07H 1/06

(57) ABSTRACT

The present application provides a method for purifying sucralose-6-ester. The method comprises: adding an oxidizing agent to a mother solution to be purified, carrying out an oxidation reaction under a first preset condition, then adding an alkaline hydrolysis agent thereto, and carrying out an alkaline hydrolysis reaction under a second preset condition to convert a target impurity into sucralose-6-ester so as to obtain an oxidized alkaline hydrolysis reaction solution; adjusting the pH value of the oxidized alkaline hydrolysis reaction solution to be neutral, keeping same for a period of time, then heating and filtering same to remove insoluble impurities, and then carrying out liquid separation to obtain a first aqueous phase and a first organic phase; extracting the first aqueous phase by using an extracting agent, and combining a second organic phase, which is obtained by means of the extraction, with the first organic phase to obtain a mother solution to be crystallized; and a step of crystallization, involving: concentrating the mother solution to be crystallized, and carrying out crystallization under a third preset condition to obtain a crude product of sucralose-6-ester and a first filtrate. By means of the present application, the yield of a target product is greatly improved while the impurity content is effectively removed.

### TECHNICAL FIELD

The invention belongs to the technical field of fine chemical engineering, and particularly relates to a method for purifying sucralose-6-ester.

### BACKGROUND

Sucralose, a novel sweetener, was first synthesized by the British company Tate & Lyte, which can achieve about 600 times the sweetness of sucrose. Sucralose has the characteristics of no energy, high sweetness, pure sweetness, high safety and the like, and is one of the most competitive sweeteners artificially synthesized so far. Its market prospect is board. At present, the widely used method for synthesizing sucralose in industry is, taking sucrose as a raw material, carrying out esterification reaction on the sucrose to prepare sucrose-6-ester, then chlorinating the sucrose-6-ester to obtain sucralose-6-ester, and finally carrying out deesterification on sucralose-6-ester to obtain sucralose.

In the prior art, sucralose-6-ester is usually purified first, and then a subsequent deesterification step is performed, the purification of sucralose-6-ester is usually carried out in an extractant, such as ethyl acetate to crystallize, and after crystallization, impurities are mainly concentrated in the mother solution. The mother solution retained after the first crystallization is called primary mother solution, the primary mother solution is concentrated, decolored and impurity-removed and then recrystallized, the mother solution retained after the second crystallization is called secondary mother solution, the secondary mother solution is concentrated, decolored and boiled to remove impurities, a small amount of crystals can be obtained, the secondary mother solution and the mother solution retained after secondary mother solution recrystallization are both called repeated mother solution.

The color of the secondary mother solution or the mother solution after secondary mother solution is relatively dark, the viscosity is high, the extraction is difficult, the method for extracting the repeated mother solution in the prior art generally adopts the conventional method of extracting by boiling for many times, then extracting the aqueous phase by using the organic extractant and recrystallizing, this method has a lot of defects: the separation efficiency is low, the solubility of the sucralose-6-ester in extractant is far greater than that in water, so sucralose-6-ester needs to be extracted for many times, and the energy consumption is high; although after many times of extraction, sucralose-6-ester still can not be completely collected, part of sucralose-6-ester can not be collected along with the treatment of the final waste solution; polychlorinated compounds generated by chlorination in the repeated mother solution, such as sucralose diester and the like, are not converted and utilized, causing a large amount of waste, increasing the pressure of environmental protection treatment.

In view of the above, a method for purifying sucralose-6-ester is needed to solve the above problems. It should be appreciated that the statements herein merely provide background information related to the present disclosure and may not necessarily constitute prior art.

### SUMMARY

In view of the prior art, the application provides a method for purifying sucralose-6-ester, which overcomes the problems or at least partially solves the problems that in a process for purifying sucralose-6-ester repeated mother solution, the defects of high energy consumption, low separation efficiency, difficult subsequent waste solution treatment, low economic value and the like exist, and the yield of sucralose-6-ester is low.

According to the first aspect of the present application, a method for purifying sucralose-6-ester is provided, comprising the following steps.

An impurity conversion step: adding an oxidant into a mother solution to be purified, carrying out oxidation reaction under a first preset condition, then adding an alkaline hydrolysis agent thereto, and carrying out alkaline hydrolysis reaction under a second preset condition to convert target impurities into sucralose-6-ester to obtain an oxidization alkaline hydrolysis reaction solution; wherein the mother solution to be purified is a mother solution crystallized repeatedly of sucralose-6-ester;

A pH adjusting step: adjusting the pH of the oxidization alkaline hydrolysis reaction solution to be neutral, keeping the pH for a period of time, then heating, filtering to remove insoluble impurities, and separating the solution to obtain a first aqueous phase and a first organic phase;

An extraction step: extracting the first aqueous phase by using an extractant, and combining a second organic phase obtained by extraction with the first organic phase to obtain a mother solution to be crystallized; and

A crystallization step: concentrating the mother solution to be crystallized, and

crystallizing under a third preset condition to obtain a crude sucralose-6-ester product and a first filtrate.

Optionally, in the above method, before the impurity convertion step, the method further comprises the following steps.

Mother solution condensation step: condensing the mother solution to be purified to −10-30° C., preferably 0-10° C.

Optionally, in the above method, the method further includes:

A refining step: dissolving the obtained crude sucralose-6-ester product in an extractant, and recrystallizing under a fourth preset condition to obtain a refined sucralose-6-ester product.

Optionally, in the above method, dissolving the obtained crude sucralose-6-ester product in an extractant, and recrystallizing under a fourth preset condition to obtain a refined sucralose-6-ester product includes the following process.

Dissolving the crude sucralose-6-ester product in an extractant with a mass ratio of 1-3:1, preferably 1.5-2:1 to the crude sucralose-6-ester product under the condition of heating, controlling the recrystallization temperature to be 5-15° C., preferably 0-5° C., and the recrystallization time to be 2-12 hours, preferably 6-10 hours, filtering and drying after crystallization is completed, to obtain a refined sucralose-6-ester product.

Optionally, in the above method, the oxidant is hydrogen peroxide;

The mass fraction of the hydrogen peroxide is 15-40%, preferably 25-35%;

The volume of the hydrogen peroxide is 5-20%, preferably 10-15% of the volume of the mother solution to be purified.

Optionally, in the method, the first preset condition is as follows.

The reaction temperature of the oxidation reaction is set to be-10 to 20° C., preferably 0 to 10° C.;

The reaction time of the oxidation reaction is set to be 1-12 h, preferably 6-10 h.

Optionally, in the above method, the alkaline hydrolysis agent is one or more of ammonia, triethylamine, diethylamine and dimethylamine; preferably, the alkaline hydrolysis agent is dimethylamine, wherein the concentration of dimethylamine is 10-45 wt %, preferably 30-40 wt %.

Optionally, in the method, the second preset condition is as follows.

The reaction temperature of alkaline hydrolysis reaction is set to be −10 to 30° C., preferably 0 to 10° C.;

The reaction time of alkaline hydrolysis reaction is set to be 3-12 h, preferably 6-10 h;

The pH of the alkaline hydrolysis reaction is 7-12, preferably 9-10.

Optionally, in the above method, in the pH adjusting step, adjusting the pH of the oxidation alkaline hydrolysis reaction solution to be neutral, keeping for a period of time, then heating and filtering to remove insoluble impurities includes the following process.

Using an acidic substance to adjust the pH of the oxidation alkaline hydrolysis reaction solution to 6-8, preferably 7, keeping for 1-2 h, heating to 30-60° C., preferably 40-50° C., and then filtering out insoluble impurities; wherein the acidic substance is sulfuric acid, acetic acid or hydrochloric acid, preferably hydrochloric acid; the mass fraction of the acidic substance is 10-35%, preferably 25-30%.

Optionally, in the above method, in the extracting step, the extractant is ethyl acetate;

The ratio of the volume of the extractant to the volume of the first aqueous phase is 0.5-4:1, preferably 1-2:1;

The content of sucralose-6-ester in the second aqueous phase obtained after extraction is lower than 1 g/L.

Optionally, in the above method, in the crystallization step, vacuum distillation is adopted to concentrate the mother solution to be crystallized, and the distillation temperature is 30-80° C., preferably 35-55° C.; the distillation pressure is from −0.050 MPa to −0.99 MPa, preferably from −0.50 MPa to −0.8 MPa; concentrating the mother solution to be crystallized to the density of 1-2 g/cm³, preferably 1.1-1.5 g/cm³.

Optionally, in the above method, in the crystallization step, the third preset condition is as follows.

The crystallization time is set to be 2-24 h, preferably 6-12 h; the crystallization temperature is set to be −10 to 15° C., preferably 0 to 5° C.

The application has the beneficial effects that the application aims at treating the repeated mother solution of sucralose-6-ester by combining the oxidant and alkaline hydrolysis agent to convert impurities such as sucralose diester and tetrachlorosucrose-6-ester into the target product sucralose-6-ester, so that the impurity content is effectively removed, and the yield of the target product is greatly improved; the whole treatment process is simple and smooth, the economic cost is low, and the practicability is strong; the purity of the obtained sucralose-6-ester is high, and the production of the sucralose can be directly carried out; the method can obviously reduce the impurity content of the wastewater, relieves the pressure of subsequent wastewater treatment, is environment- friendly, and has extremely high application and economic values.

The above description is only an overview of the technical solutions of the present application, and the present application can be implemented in accordance with the content of the description so as to make the technical means of the present application more clearly understood, and the above and other objects, features, and advantages of the present application will be more clearly understood.

### BRIEF DESCRIPTION OF FIGURES

Various additional advantages and benefits will become apparent to those of ordinary skill in the art upon reading the following detailed description of the preferred embodiments. The drawings are only for purposes of illustrating the preferred embodiments and are not to be construed as limiting the application. Also, like reference numerals are used to refer to like parts throughout the drawings.

FIG. 1 shows a schematic flow diagram of a method for purifying sucralose-6-ester according to one embodiment of the application.

### DETAILED DESCRIPTION

Exemplary embodiments of the present application will be described in more detail below. While exemplary embodiments of the application are shown, it should be understood that the application may be embodied in various forms and should not be limited to the embodiments set forth herein. Rather, these embodiments are provided so that this disclosure will be thorough and complete, and will fully convey the scope of the application to those skilled in the art.

The application is characterized in that the sucralose-6-ester is obtained by chlorinating the sucrose-6-ester by phosgene or thionyl chloride, and in the prior art, the chlorination conversion rate is about 60-70%, so that the yield of the sucralose-6-ester needs to be improved as much as possible in order to improve the yield of the sucralose.

In the prior art, the method for purifying the repeated mother solution of sucralose-6-ester has the defects of high energy consumption, low separation efficiency, difficult subsequent waste liquid treatment, low economic value and the like, and the yield of the sucralose-6-ester is very low. In view of the situation, the application provides a method for purifying sucralose-6-ester, which combines oxidant and alkaline hydrolysis agent to convert target impurities such as sucralose diester and tetrachlorosucrose-6-ester in the repeated mother solution of sucralose-6-ester into the target product sucralose-6-ester, so that the impurity content is effectively removed, and the yield of the sucralose-6-ester is remarkably improved.

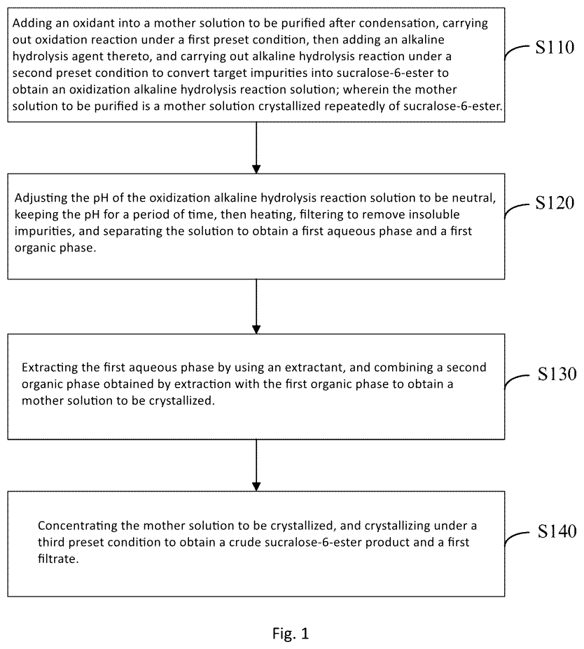

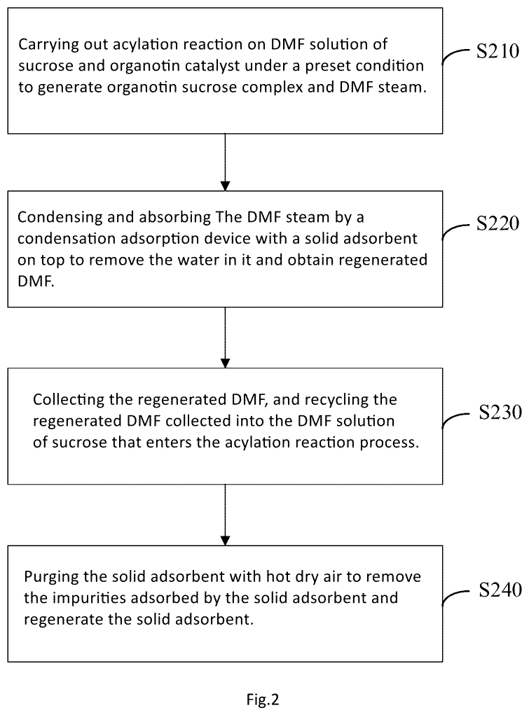

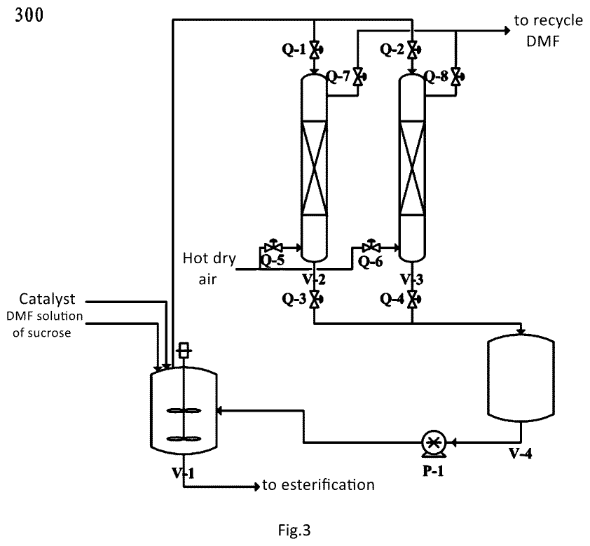

FIG. 1 shows a schematic flow diagram of a method for purifying sucralose-6-ester according to an embodiment of the present application, and as can be seen from FIG. 1, the method at least comprises steps S110 to S140.

Impurity conversion step S110: adding an oxidant into a mother solution to be purified, carrying out oxidation reaction under a first preset condition, then adding an alkaline hydrolysis agent into the mother solution, and carrying out alkaline hydrolysis reaction under a second preset condition to convert target impurities into sucralose-6-ester to obtain an oxidation alkaline hydrolysis reaction solution; wherein the mother solution to be purified is a mother solution crystallized repeatedly of sucralose-6-ester.

The mother solution to be purified in the application refers to the mother solution in which the chlorination reaction solution of sucrose-6-ester is retained after at least more than one time crystallization, namely the reactant mixed solution obtained by the chlorination reaction of the sucrose-6-ester and a chlorination agent, and the reactant mixed solution is crystallized for one time to obtain part of sucrose-6-ester crude product and a primary mother solution; recrystallizing the primary mother solution again to obtain part of sucrose-6-ester crude product and a secondary mother solution; and recrystallizing the secondary mother solution to obtain part of sucrose-6-ester crude product and a tertiary mother solution, wherein the secondary mother solution, the tertiary mother solution and further mother solution can all be treated by the application and are called as mother solution crystallized repeatedly.

Since strong chlorination agents such as thionyl chloride and phosgene are used in the chlorination reaction process, a series of byproducts such as monochlorosucrose-6-carboxylate, dichlorosucrose-6-carboxylate, tetrachlorosucrose-6-carboxylate, sucralose diester and the like are generated due to high temperature in the chlorination incubation stage, and organic impurities such as N, N-Dimethylformamide (DMF) and trichloroethane, and a large amount of water are contained. At present, in the prior art, multiple boiling extraction is usually adopted for treating sucrose-6-ester repeated mother solution, and then organic extractant is adopted for extracting a aqueous phase and recrystallizing, this method is complex, the energy consumption is high, the efficiency is low, a large amount of waste liquid is generated, and the economic value is low. Taking the sucralose-6-ester as an example of the sucralose-6-ethyl ester, the applicant finds that the mother solution crystallized repeatedly contains more sucralose diester, tetrachlorosucrose-6-ethyl ester, and less monochlorospyrose-6-ethyl ester and dichlorosucrose-6-ethyl ester besides the target sucralose-6-ethyl ester through analysis on the mother solution crystallized repeatedly. Researches show that sucralose diester and tetrachlorosucrose-6-ethyl ester can be selectively oxidized to obtain the target product sucralose-6-ethyl ester, so that the impurities are removed, and the yield of the target product sucralose-6-ethyl ester is improved.

After the oxidant is added, the oxidant and sucralose diester, tetrachlorosucrose-6-ethyl ester are subjected to oxidation reaction to generate a composite intermediate. After the oxidation reaction is carried out for a certain period of time, an alkaline hydrolysis agent is added thereto. After the oxidant is added, on one hand, the pH of the whole aqueous solution to be purified can be changed, and particularly, an acid environment can be formed; on the other hand, the target impurities of sucralose diester and tetrachlorosucrose-6-ester and the oxidant can form corresponding composite intermediates, and the pH of the composite intermediates is adjusted to 8.5-10.5 by alkaline substances, because the composite intermediates formed by the oxidant and the target impurities of sucralose diester and tetrachlorosucrose-6-ester need alkaline substances to neutralize groups to be removed, so that the target product of sucralose-6-ester can be formed.

Taking hydrogen peroxide as an oxidant and dimethylamine as an alkaline hydrolysis agent as an example, the chemical reaction formula for converting the impurities of sucralose diester and tetrachlorosucrose-6-ester into the target product sucralose-6-ester is as follows.

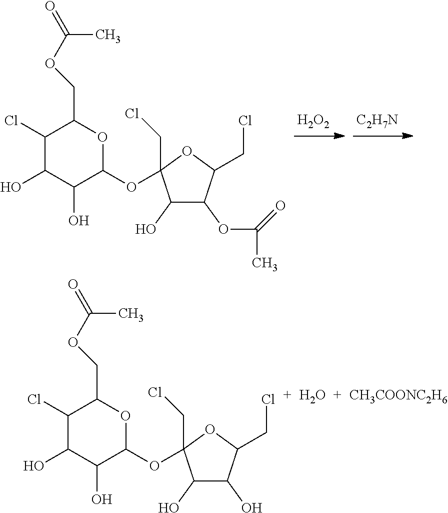

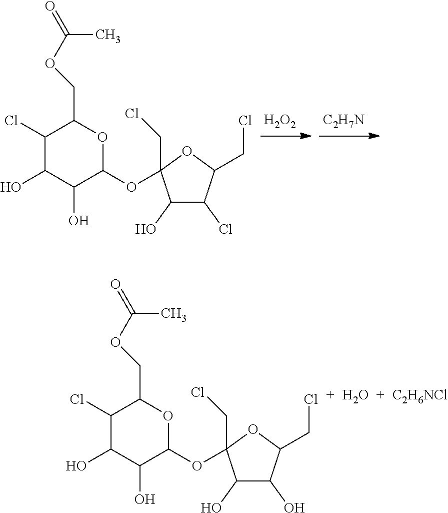

According to the chemical reaction formula, the reaction of the oxidant on the sucralose diester in the presence of the alkaline hydrolysis agent is to selectively remove the ester group to generate the sucralose-6-ester; the reaction of the oxidant on the tetrachlorosucrose-6-ester is to selectively remove one chlorine atom to generate the trichlorosucrose-6-ester.

pH adjusting step S120: adjusting the pH of the oxidation alkaline hydrolysis reaction solution to be neutral, keeping the pH for a period of time, then heating, filtering to remove insoluble impurities, and separating the solution to obtain a first aqueous phase and a first organic phase;

After the mother solution to be purified is subjected to oxidation reaction, the pH can be influenced, the obtained oxidation alkaline hydrolysis reaction solution is alkaline, and before crystallization is carried out, the solution is preferably adjusted to be neutral, an acidic substance can be used for adjusting the solution to be central. In the present application, “neutral” is not a condition that the pH is absolutely equal to 7, it could be considered to be neutral when the pH is about 7, for example, the pH is in the range of 6 to 8. In the condition process, false neutrality may exist, namely the pH is measured to be 6-8, but after a while, the pH becomes acidic, therefore, subsequent steps are continued after the pH is stabilized at 6-8 for a while.

After the pH adjusting is completed, the entire mass can be warmed to completely dissolve the soluble materials in the mass, while the insoluble impurities can be removed by filtration. The sucralose-6-ethyl ester is prepared by using sucrose as a raw material, side reactions occur among sucrose in the reaction process, sucrose molecules are polymerized and coked to form colored insoluble impurities, and the insoluble impurities can be removed by dissolution and filtration. After filtration, liquid separation treatment is carried out to obtain a first aqueous phase and a first organic phase.

The vast majority of organic esters, including the target product sucralose-6-ester, as well as monochloro sucrose-6-ethyl ester and dichlorosucrose-6-ethyl ester, are distributed primarily in the first organic phase; while a small portion of the organic esters, still including the target sucralose-6-ester, as well as monochlorosucrose-6-ethyl ester and dichlorosucrose-6-ethyl ester, are distributed in the first aqueous phase.

An extraction step S130: extracting the first aqueous phase by using an extractant, and combining a second organic phase obtained by extraction with the first organic phase to obtain the mother solution to be crystallized.

Extracting the first aqueous phase by using an extractant, so that the organic ester originally distributed in the first aqueous phase is extracted into the extractant, extracting and separating to obtain the second organic phase, dissolving most of the organic ester originally distributed in the first aqueous phase into the second organic phase, and combining the second organic phase with the first organic phase to obtain the mother solution to be crystallized.

And, a crystallization step S140: concentrating the mother solution to be crystallized, and crystallizing under a third preset condition to obtain a crude sucralose-6-ester product and a first filtrate.

Finally, crystallizing the mother solution to be crystallized to obtain a crude sucralose-6-ester product, the crystallization can refer to any one or combination of more of the prior art.

The obtained crude sucralose-6-ester product is high in purity, and can directly enter the next process flow for producing sucralose by the deesterification of the sucralose-6-ester.

As can be seen from the method shown in FIG. 1, the application aims at treating the repeated mother solution of sucralose-6-ester by combining the oxidant and alkaline hydrolysis agent to convert impurities such as sucralose diester and tetrachlorosucrose-6-ester into the target product sucralose-6-ester, so that the impurity content is effectively removed, and the yield of the target product is greatly improved; the whole treatment process is simple and smooth, the economic cost is low, and the practicability is strong; the purity of the obtained sucralose-6-ester is high, and the production of the sucralose can be directly carried out; the method can obviously reduce the impurity content of the wastewater, relieves the pressure of subsequent wastewater treatment, is environment-friendly, and has extremely high application and economic values.

In some embodiments of the application, prior to the impurity convertion step, the method further comprises mother solution condensation step: condensing the mother solution to be purified to −10-30° C., preferably 0-10° C.

The chlorination reaction is usually above 60° C., the oxidation reaction is a violent exothermic reaction, and in view of safety and reaction efficiency, the mother solution to be purified can be firstly condensed, specifically, the mother solution to be purified is condensed to −10-30° C., preferably 0-10° C. If the condensation temperature is below −10° C., a large amount of additional condensing medium and energy consumption are needed, resulting in a sudden increase in costs and no other beneficial effects; if the condensation temperature is above 30° C., the reaction is not favorable for forward direction.

In some embodiments of the application, the method further comprises a refining step: and dissolving the obtained crude sucralose-6-ester product in an extractant, and recrystallizing under a fourth preset condition to obtain a refined sucralose-6-ester product.

After the primary crystallization step, the obtained sucralose-6-ester has low purity, and the sucralose-6-ester with higher purity can be obtained by a recrystallization method. The recrystallization can adopt any one or combination of more of the prior art, or the crude sucralose-6-ester product can be dissolved in an extractant, and the obtained extract is recrystallized under a fourth preset condition to obtain the sucralose-6-ester refined product. Specifically, in some embodiments of the present application, the crude sucralose-6-ester product is dissolved in an extractant at a mass ratio of 1-3:1 to the crude sucralose-6-ester product under a heating condition, for example, heating to 65° C., and the recrystallization temperature is controlled to be 5-15° C.; recrystallization time to be 2-12 h for recrystallization; in other embodiments, the crude sucralose-6-ester product is dissolved in an extractant with the mass ratio of 1.5-2:1 to the crude sucralose-6-ester product, the recrystallization temperature is controlled to be 0-5° C., the recrystallization time to be 6-10 h for recrystallization; after crystallization is finished, filtering and drying are carried out to obtain the refined sucralose-6-ester product.

The Type of Oxidant

In some embodiments of the present application, the oxidant is screened, and when screening the oxidants, it was found that when hydrogen peroxide is selected, the selective oxidizing property is better, that is, sucralose diester and tetrachlorosucrose-6-ester can be oxidized to sucralose-6-ester, while sucralose-6-ester will not be further oxidized.

In some embodiments of the application, the mass fraction and the amount of the hydrogen peroxide are not limited, and in other embodiments, the mass fraction of the hydrogen peroxide is 15-40%, and the volume amount of the hydrogen peroxide is 5-20% of the volume of the mother solution to be purified; in other embodiments, the mass fraction of the hydrogen peroxide is 25-35%, and the volume of the hydrogen peroxide is 10-15% of the volume of the mother solution to be purified.

### Oxidation Reaction Condition (First Preset Condition)

In some embodiments of the present application, the temperature and time of the oxidation reaction are not limited, and the range of the oxidation reaction in the prior art can be referred to, so as to ensure complete reaction; in other embodiments, the temperature of the oxidation decoloring reaction is −10-20° C. and the reaction time is 1-12 h, and in still other embodiments, the temperature of the oxidation decoloring reaction is 0-10° C. and the reaction time is 6-10 h; if the reaction temperature is lower than −10° C. and the reaction time is shorter than 1 h, the reaction condition is mild, and the problem of incomplete oxidation reaction is easily caused; if the reaction temperature is higher than 20° C. and the reaction time is longer than 12 h, excessive reaction may occur, and sucralose-6-ester may be oxidized together.

The amount and type of alkaline hydrolysis agent

In some embodiments of the present application, the amount of the alkaline hydrolysis agent is not limited as long as alkaline environment is achieved, in other embodiments, the amount of the alkaline hydrolysis agent is based on adjusting the pH of the oxidation alkaline hydrolysis reaction solution to 8.5-10.5.

In some embodiments of the present application, the type of the alkaline hydrolysis agent is not limited, any alkaline substance is possible, and in other embodiments, the alkaline hydrolysis agent is one or more of ammonia, triethylamine, diethylamine, and dimethylamine; in still other embodiments, the alkaline hydrolysis agent is dimethylamine, wherein the concentration of dimethylamine is 10%-45 wt%, and in still other embodiments, the concentration of dimethylamine is 30%-40 wt%.

Dimethylamine recovered from the previous steps can be used as the dimethylamine herein, a large amount of N, N-Dimethylformamide (DMF) is used in the preparation process of sucrose-6-ester, the N, N-dimethylformamide can be decomposed to generate dimethylamine in the reaction process, and a large amount of dimethylamine can be recovered in the solvent recovery process. This part of dimethylamine can be used as an alkaline hydrolysis agent, so that the material utilization of the whole production flow is optimized, and the purification cost of sucralose-6-ester is greatly reduced.

Reaction condition of alkaline hydrolysis reaction (second preset condition)

In some embodiments of the present application, the reaction condition of alkaline hydrolysis reaction is not limited, and all that is needed is to remove the group to be removed of the composite intermediate generated by the oxidation reaction of the oxidant and sucralose diester, tetrachlorosucrose-6-ethyl ester; in other embodiments of the present application, the reaction temperature of the alkaline hydrolysis reaction is −10° C.-30° C., the reaction time of the alkaline hydrolysis reaction is set to be 3-12 h, and the pH of the alkaline hydrolysis reaction is set to be 7-12; in other embodiments, the reaction temperature of the alkaline hydrolysis reaction is 0-10° C., the reaction time of the alkaline hydrolysis reaction is set to be 6-10 h, and the pH of the alkaline hydrolysis reaction is 9-10. If the reaction temperature of the alkaline hydrolysis reaction is lower than −10° C., the reaction time is less than 3 h, and the pH is lower than 7, the condition is too mild, and the groups to be removed cannot be completely removed, so that the conversion of target impurities is influenced; if the reaction temperature of the dealkalization reaction is higher than 30° C., the reaction time is more than 12 h, and the pH is higher than 12, the reaction condition is too violent, so that not only the target impurities are oxidized, but also the sucralose-6-ester can be oxidized.

### pH Adjusting Condition

In some embodiments of the present application, the method and condition for adjusting the pH are not limited, the value can be adjusted by acidic substance; in other embodiments, the pH of the oxidation alkaline hydrolysis reaction solution is adjusted to 6-8 by using acidic substance, keeping for 1-2 h, then the separation of insoluble impurities is carried out, specifically, the temperature can be raised to 30-60° C. firstly, and then the insoluble impurities are filtered; wherein the acidic substance is sulfuric acid, acetic acid or hydrochloric acid; the mass fraction of the acidic substance is 10%-35%. In still other embodiments, hydrochloric acid with the mass fraction of 25%-30% is used to adjust the pH of the oxidation alkaline hydrolysis reaction solution to 7, keeping for 1-2 h, then the temperature is raised to 40-50° C., and insoluble impurities are filtered. Wherein, the hydrochloric acid can be obtained by decomposing and recycling a chlorinated reagent thionyl chloride, and the use of the hydrochloric acid can obviously reduce the economic cost.

### The Type and Amount of the Extractant

In some embodiments of the application, the type and amount of the extractant are not limited, as long as the organic esters can be effectively dissolved; in other embodiments of the application, the extractant is one or a mixture of methyl acetate, ethyl acetate, propyl acetate, isopropyl acetate, butyl acetate, and isobutyl acetate, and in still other embodiments is ethyl acetate. Since the target product sucralose-6-ester and the impurities monochloro sucrose-6-ester, dichloro sucrose-6-ester, sucralose diester and tetrachloro sucrose-6-ester all can be dissolved in the above esters and water, but the solubility in the extractant is higher than in water, so that the organic ester impurities in the target product can be extracted from the reaction solution.

In some embodiments of the application, the amount of the extractant is not limited, as long as the extraction requirement can be met, and in other embodiments, the ratio of the volume of the extractant to the volume of the first aqueous phase is 0.5-4:1 for economic reasons; in still other embodiments, the ratio of the volume of the extractant to the volume of the first aqueous phase is 1-2:1.

In order to ensure the yield of sucralose-6-ester, in some embodiments of the application, the content of sucralose-6-ester in the second aqueous phase obtained after extraction is less than 1 g/L, which can ensure that most sucralose-6-ester is extracted into the extractant. Sucralose-6-ester can be tested by high performance liquid chromatography.

### Crystallization Condition (Third Preset Condition)

In some embodiments of the present application, the crystallization condition is not limited, the prior art can be refered to; in other embodiments, the mother solution to be crystallized can be concentrated first, for example, the mother solution to be crystallized is concentrated by reduced pressure distillation, wherein the distillation temperature of the distillation is 30-80° C.; the distillation pressure is −0.050 MPa to −0.99 MPa, the mother solution to be crystallized is concentrated to a density of 1˜2 g/cm³, and stopped concentration. In still other embodiments, the mother solution to be crystallized is concentrated to a density of 1.1-1.5 g/cm³ at a distillation temperature of 35-55° C. and a distillation pressure of-0.50 MPa to-0.8 MPa, and then crystallization is carried out.

In some embodiments of the present application, the crystallization time and crystallization temperature are not limited, and in other embodiments, the crystallization time is set to be 2-24 h, and the crystallization temperature is set to be −10-15° C.; in still other embodiments, the crystallization time is set to be 6-12 h, and the crystallization temperature is set to be 0-5° C. The crystallization condition can influence the yield and the purity of the crystallized product, the above crystallization condition is screened by the inventor through a large number of experiments, as for the crystallization conditions of the repeated mother solution of the sucralose-6-ester, can give consideration to both the yield and the purity of the sucralose-6-ester, thereby achieving a relatively ideal effect.

### Test Methods Involved in the Application

The contents of the substances in the examples and comparative examples of the present application were measured by High Performance Liquid Chromatography (HPLC) under the following conditions using an external standard method, and are not described in detail in the examples.

Analytical measurement conditions for high performance liquid chromatography: Japan Shimadzu high performance liquid chromatograph, RID-10A refractive index detection unit, LC-10ADVP high-pressure pump, CTO-10ASVP constant temperature box equipped; chromatographic column: Agilent XDB C18 column (250 mm×4.6 mm, 5 μm); mobile phase: methanol-0.125% dipotassium hydrogen phosphate aqueous solution (4:6); column temperature: 30° C.; flow rate: 1.0 mL/min. Wherein, methanol (chromatogram purity), dipotassium hydrogen phosphate (analytical purity), ultrapure water and the like are needed, and the content is measured by an external standard method for each standard substance.

### Example 1

(a) 1000 mL of secondary mother solution of sucralose-6-acetate (the initial concentration of the sucralose-6-acetate is 32 g/L) is added into a 2000 mL three-neck flask, and the temperature is reduced to 2+/−2° C. Dropwise adding hydrogen peroxide with the mass fraction of 27.5% for oxidation reaction, wherein the dosage of the hydrogen peroxide is 120 mL, and keeping the temperature for 6 hours after dropwise adding the hydrogen peroxide, wherein the temperature is kept at about 5° C.
(b) After the reaction of the materials in the step (a) is finished, adding 125 mL of dimethylamine with the mass fraction of 40% to perform alkaline hydrolysis reaction, controlling the reaction temperature to be about 5° C., controlling the pH of the reaction to be about 9, after the pH is stable, keeping the temperature and maintaining the alkaline hydrolysis time to be about 10 hours.
(c) Adding hydrochloric acid with the mass fraction of 30% into the reaction solution obtained in the step (b), adjusting the pH back to about pH=7.5, the amount of the hydrochloric acid is about 12 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining the washed ethyl acetate with the layered first organic phase, and carrying out reduced pressure distillation and concentration under the conditions that the temperature is controlled at 50° C. and the pressure is controlled at −0.06 MPa. Concentrating the specific gravity of the mother solution to be crystallized to about 1.5 g/cm³, stopping concentration, then cooling to about 0° C., and stirring for 12 hours to complete crystallization.

51.28 g of pure sucralose-6-acetate product with a purity of 97.06% is obtained after the reaction, which can be directly used for sucralose production, the yield is greatly improved compared with 32 g of initially-added sucralose-6-acetate product, liquid phase analysis can show that polychlorinated compounds and sucralose diester obviously disappeared after the reaction, showing that the reaction is complete and the conversion is complete. The corresponding effect on yield is obvious.

### Example 2

(a) 1000 mL of secondary mother solution of sucralose-6-acetate (the initial concentration of the sucralose-6-acetate is 32 g/L) is added into a 2000 mL three-neck flask, and the temperature is reduced to 2+/−2° C. Dropwise adding hydrogen peroxide with the mass fraction of 27.5% for oxidation reaction, wherein the dosage of the hydrogen peroxide is 120 mL, and keeping the temperature for 6 hours after dropwise adding the hydrogen peroxide, wherein the temperature is kept at about 5° C.
(b) After the reaction of the materials in the step (a) is finished, adding 125 mL of dimethylamine with the mass fraction of 40% to perform alkaline hydrolysis reaction, controlling the reaction temperature to be about 5° C., controlling the pH of the reaction to be about 9, after the pH is stable, keeping the temperature and maintaining the alkaline hydrolysis time to be about 10 hours.
(c) Adding hydrochloric acid with the mass fraction of 30% into the reaction solution obtained in the step (b), adjusting the pH back to about pH=7.5, wherein the amount of the hydrochloric acid is about 12 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of first aqueous phase and a first organic phase after layering, adding 250 mL of ethyl acetate into the first aqueous phase for extraction once, combining the washed ethyl acetate with the layered first organic phase, and carrying out reduced pressure distillation and concentration under the conditions that the temperature is controlled at 50° C. and the pressure is controlled at −0.06 MPa. Concentrating the specific gravity of the mother solution to be crystallized to about 1.5 g/cm³, stopping concentration, then cooling to about 0° C., and stirring for 12 hours to complete crystallization.
(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 5° C., recrystallization time is controlled to be 12 hours, and drying the filter cake after filtration.

53.28 g of pure sucralose-6-acetate with a purity of 98.76% is obtained after the reaction, which can be directly used for sucralose production, the yield is greatly improved compared with 32 g of initially-added sucralose-6-acetate, and the polychlorinated compounds and sucralose diester which can be obtained through liquid phase analysis obviously disappear after the reaction, which indicates that the reaction is relatively sufficient and the conversion is relatively complete. The corresponding effect on yield is obvious.

### Example 3

(a) 1000 mL of secondary mother solution of sucralose-6-acetate (the initial concentration of the sucralose-6-acetate is 32 g/L) is added into a 2000 mL three-neck flask, and the temperature is reduced to −10° C. Dropwise adding hydrogen peroxide with the mass fraction of 40% for oxidation reaction, wherein the dosage of the hydrogen peroxide is 50 mL, and keeping the temperature for 12 hours after dropwise adding the hydrogen peroxide, wherein the temperature is kept at about 5° C.
(b) After the reaction of the materials in the step (a) is finished, adding 150 mL of dimethylamine with the mass fraction of 30% to perform alkaline hydrolysis reaction, controlling the reaction temperature to be about 5° C., controlling the pH of the reaction to be about 9, after the pH is stable, keeping the temperature and maintaining the alkaline hydrolysis time to be about 12 hours.
(c) Adding hydrochloric acid with the mass fraction of 15% into the reaction solution obtained in the step (b), adjusting the pH back to about pH=7.0, wherein the amount of the hydrochloric acid is about 22 mL, measuring the pH again and keeping the pH basically stable after the adjustment is finished and maintained for 60 minutes.
(d) Heating the reacted material to 60° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining washed ethyl acetate with the layered first organic phase, and carrying out reduced pressure distillation and concentration under the conditions that the temperature is controlled at 50° C. and the pressure is controlled at −0.099 MPa. Concentrating the specific gravity of the mother solution to be crystallized to about 1.5g/cm³, stopping concentration, then cooling to about 0° C., and stirring for 24 hours to complete crystallization.
(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 5° C., recrystallization time is controlled to be 2 hours, and drying filter cake after filtration.

52.28 g of pure sucralose-6-acetate with a purity of 97.11% is obtained after the reaction, which can be directly used for sucralose production, the yield is greatly improved compared with 32 g of initially-added sucralose-6-acetate, and the polychlorinated compounds and sucralose diester which can be obtained through liquid phase analysis obviously disappear after the reaction, which indicates that the reaction is relatively sufficient and the conversion is relatively complete. The corresponding effect on yield is obvious.

### Example 4

(a) 1000 mL of the secondary mother solution of sucralose-6-acetate (the initial concentration of sucralose-6-acetate is 32 g/L) is added into a 2000 mL three-necked flask, and the temperature is reduced to 30° C. Dropwise adding hydrogen peroxide with the mass fraction of 27.5% for oxidation reaction, wherein the dosage of the hydrogen peroxide is 200 mL, and keeping the temperature for 6 hours after dropwise adding the hydrogen peroxide, wherein the temperature is kept at about 20° C.
(b) After the reaction of the materials in the step (a) is finished, adding 140 mL of dimethylamine with the mass fraction of 40% to perform alkaline hydrolysis reaction, controlling the reaction temperature to be about 5° C., controlling the pH of the reaction to be about 9, after the pH is stable, keeping the temperature and maintaining the alkaline hydrolysis time to be about 6 hours.
(c) Adding hydrochloric acid with the mass fraction of 30% into the reaction solution obtained in the step (b), adjusting the pH back to about pH=7.5, wherein the amount of the hydrochloric acid is about 15 mL, measuring the pH again and keeping the pH basically stable after the adjustment is finished and maintained for 120 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining the washed ethyl acetate with the layered first organic phase, and carrying out reduced pressure distillation and concentration under the conditions that the temperature is controlled at 50° C. and the pressure is controlled at −0.06 MPa. Concentrating the specific gravity of the mother solution to be crystallized to about 1.8 g/cm³, stopping concentration, then cooling to about 0° C., and stirring for 12 hours to complete crystallization.
(e) Filtering the material crystallized in the step (d), and adding ethyl acetate with the volume 3 times that of the filter cake into the filter cake. Heating to 60° C., cooling for recrystallization after stirried to dissolution, the recrystallization temperature is controlled to be about 5° C., recrystallization time is controlled to be 12 hours, and drying filter cake after filtration.

52.66 g of pure sucralose-6-acetate with a purity of 98.56% is obtained after the reaction, which can be directly used for sucralose production, the yield is greatly improved compared with 32 g of initially-added sucralose-6-acetate, and the polychlorinated compounds and sucralose diester which can be obtained through liquid phase analysis obviously disappear after the reaction, which indicates that the reaction is relatively sufficient and the conversion is relatively complete. The corresponding effect on yield is obvious.

### Example 5

(a) 1000 mL of the secondary mother solution of sucralose-6-acetate (the initial concentration of the sucralose-6-acetate is 32 g/L) is added into a 2000 mL three-neck flask, and the temperature is reduced to about 10° C. Dropwise adding hydrogen peroxide with the mass fraction of 27.5% for oxidation reaction, wherein the dosage of the hydrogen peroxide is 150 mL, and keeping the temperature for 12 hours after dropwise adding the hydrogen peroxide, wherein the temperature is kept at about 10° C.
(b) After the reaction of the materials in the step (a) is finished, adding 150 mL of dimethylamine with the mass fraction of 30% to perform alkaline hydrolysis reaction, controlling the reaction temperature to be about 5° C., controlling the pH of the reaction to be about 9, after the pH is stable, keeping the temperature and maintaining the alkaline hydrolysis time to be about 12 hours.
(c) Adding hydrochloric acid with the mass fraction of 30% into the reaction solution obtained in the step (b), adjusting the pH back to about pH=7.5, wherein the amount of the hydrochloric acid is about 15 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining washed ethyl acetate with the layered first organic phase, and carrying out reduced pressure distillation and concentration under the conditions that the temperature is controlled at 60° C. and the pressure is controlled at −0.05 MPa. Concentrating the specific gravity of the mother solution to be crystallized to about 1.5g/cm³, stopping concentration, then cooling to about 0° C., and stirring for 12 hours to complete crystallization.
(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 10° C., recrystallization time is controlled to be 12 hours, and drying filter cake after filtration.

52.81 g of pure sucralose-6-acetate with a purity of 97.88% is obtained after the reaction, which can be directly used for sucralose production, the yield is greatly improved compared with 32 g of initially-added sucralose-6-acetate, and the polychlorinated compounds and sucralose diester which can be obtained through liquid phase analysis obviously disappear after the reaction, which indicates that the reaction is relatively sufficient and the conversion is relatively complete. The corresponding yield effect is obvious.

### Comparative Example 1

(a) 1000 mL of secondary mother solution of sucralose-6-acetate (the initial concentration of the sucralose-6-acetate is 32 g/L) is added into a 2000 mL three-neck flask, and the temperature is reduced to 2+/−2° C. Dropwise adding hydrogen peroxide with the mass fraction of 27.5% for oxidation reaction, wherein the dosage of the hydrogen peroxide is 80 mL, and keeping the temperature for 6 hours after dropwise adding the hydrogen peroxide, wherein the temperature is kept at about 5° C.
(b) After the reaction of the materials in the step (a) is finished, adding 120 mL of dimethylamine with the mass fraction of 40% to perform alkaline hydrolysis reaction, controlling the reaction temperature to be about 5° C., controlling the pH of the reaction to be about 9, after the pH is stable, keeping the temperature and maintaining the alkaline hydrolysis time to be about 10 hours.
(c) Adding hydrochloric acid with the mass fraction of 30% into the reaction solution obtained in the step (b), adjusting the pH back to about pH=7.5, wherein the amount of the hydrochloric acid is about 12 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining the washed ethyl acetate with the layered first organic phase, and carrying out reduced pressure distillation and concentration under the conditions that the temperature is controlled at 50° C. and the pressure is controlled at −0.06 MPa. Concentrating the specific gravity of the mother solution to be crystallized to about 1.5g/cm³, stopping concentration, then cooling to about 0° C., and stirring for 12 hours to complete crystallization.
(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 5° C., recrystallization time is controlled to be 12 hours, and drying filter cake after filtration.

43.28 g of pure sucralose-6-acetate with a purity of 97.63% is obtained after the reaction, which can be directly used for sucralose production, the yield is greatly improved compared with 32 g of initially-added sucralose-6-acetate, but compared with examples 2 to 5, in comparative example 1, polychlorinated compound and sucralose diester and the like are partially not reacted completely after the reaction through liquid phase analysis, which indicates that when the addition of hydrogen peroxide is too small, alkaline hydrolysis is insufficient, so that the reaction is greatly influenced, and the yield of the target product sucralose-6-acetate is low.

### Comparative Example 2

(a) 1000 mL of secondary mother solution of sucralose-6-acetate (the initial concentration of the sucralose-6-acetate is 32 g/L) is added into a 2000 mL three-neck flask, and the temperature is reduced to 2+/−2° C. Dropwise adding hydrogen peroxide with the mass fraction of 27.5% for oxidation reaction, wherein the dosage of the hydrogen peroxide is 120 mL, and keeping the temperature for 6 hours after dropwise adding the hydrogen peroxide, wherein the temperature is kept at about 5° C.
(b) After the reaction of the materials in the step (a) is finished, adding 85 mL of dimethylamine with the mass fraction of 40% to perform alkaline hydrolysis reaction, controlling the reaction temperature to be about 5° C., controlling the pH of the reaction to be about 7, after the pH is stable, keeping the temperature and maintaining the alkaline hydrolysis time to be about 10 hours.
(c) Adding hydrochloric acid with the mass fraction of 30% into the reaction solution obtained in the step (b), adjusting the pH back to about pH=7.5, wherein the amount of the hydrochloric acid is about 12 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining the washed ethyl acetate with the layered first organic phase, and carrying out reduced pressure distillation and concentration under the conditions that the temperature is controlled at 50° C. and the pressure is controlled at −0.06 MPa. Concentrating the specific gravity of the mother solution to be crystallized to about 1.5 g/cm³, stopping concentration, then cooling to about 0° C., and stirring for 12 hours to complete crystallization.
(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 5° C., recrystallization time is controlled to be 12 hours, and drying filter cake after filtration.

44.35 g of pure sucralose-6-acetate with a purity of 98.76% is obtained after the reaction, which can be directly used for sucralose production, the yield is greatly improved compared with 32 g of initially-added sucralose-6-acetate, but compared with examples 2 to 5, in comparative example 2, polychlorinated compound and sucralose diester are partially not reacted completely after the reaction through liquid phase analysis, which indicates that when the addition of dimethylamine is too small, the alkaline hydrolysis is insufficient, so that the reaction is greatly influenced, and the yield of the target product sucralose-6-acetate is low.

### Comparative Example 3

(a) 1000 mL of the secondary mother solution of sucralose-6-acetate (the initial concentration of sucralose-6-acetate is 32 g/L) is added into a 2000 mL three-necked flask, and the temperature is reduced to 0° C. Dropwise adding hydrogen peroxide with the mass fraction of 27.5% for oxidation reaction, wherein the dosage of the hydrogen peroxide is 120 mL, and keeping the temperature for 12 hours after dropwise adding the hydrogen peroxide, wherein the temperature is kept at about 5° C.
(b) After the reaction of the materials in the step (a) is finished, adding 180 mL of dimethylamine with the mass fraction of 35% to perform alkaline hydrolysis reaction, controlling the reaction temperature to be about 5° C., controlling the pH of the reaction to be about 11, after the pH is stable, keeping the temperature and maintaining the alkaline hydrolysis time to be about 10 hours.
(c) Adding hydrochloric acid with the mass fraction of 30% into the reaction solution obtained in the step (b), adjusting the pH back to about pH=7.5, wherein the amount of the hydrochloric acid is about 40 mL, measuring the pH again and keeping the pH basically stable after the adjustment is finished and maintained for 60 minutes.
(d) Heating the reacted material to 30° C., filtering to remove insoluble impurities after alkaline hydrolysis, and pouring the material into a funnel for layering. Obtaining 220 mL of a first aqueous phase and a first organic phase after layering, adding 250 mL of ethyl acetate into the first aqueous phase for extraction once, combining washed ethyl acetate with the layered first organic phase, and carrying out reduced pressure distillation and concentration under the conditions that the temperature is controlled at 60° C. and the pressure is controlled at −0.55 MPa. Concentrating the specific gravity of the mother solution to be crystallized to about 1.5 g/cm³, stopping concentration, then cooling to about 0° C., and stirring for 12 hours to complete crystallization.

(e) Filtering the material crystallized in the step (d), and adding ethyl acetate with the volume equal to that of the filter cake into the filter cake. Heating to 60° C., cooling for recrystallization after stirried to dissolution, the recrystallization temperature is controlled to be about 5° C., recrystallization time is controlled to be 12 hours, and drying filter cake after filtration.

50.66 g of pure sucralose-6-acetate with a purity of 98.64% is obtained after the reaction, the yield is greatly improved compared with 32 g of initially-added sucralose-6-acetate, polychlorinated compound and sucralose diester disappeared apparently after the reaction through liquid phase analysis, which indicates that the reaction is relatively sufficient and the conversion is relatively complete. It was also found in the experiment that the comparison of comparative example 3 and examples 2 to 5 resulted in: comparative example 3 has a great increase in sucralose content, which indicates that

alkaline hydrolysis under peralkaline conditions will cause a hydrolysis reaction of sucralose-6-acetate, and the hydrolyzed sucralose is difficult to collect by an extraction method, so that the overall yield is significantly affected. Indicating that pH has a side effect on the reaction under the condition of peralkalinity.

### Comparative Example 1

(a) 1000 mL of a secondary mother solution of sucralose-6-acetate (the initial concentration of sucralose-6-acetate is 32 g/L) is added into a 3000 mL three-necked flask, equal volume of hot water is added at about 80° C., stirring and heating to maintain the temperature at about 80° C. After stirring for 1 hour, the mixture is separated into layers by a separatory funnel, and the aqueous phase is kept for further use.
(b) Adding equal volume of 80° C. hot water into the organic phase after layering, continuing the process of the step (a), and combining the boiled aqueous phases twice. The ester phase is concentrated to recover ethyl acetate, and the residual sugar residue is used as waste material for treatment.
(c) The twice combined aqueous phase is concentrated to a volume of 500 mL under −0.9 MPa at 80° C. and extracted three times with equal amount of ethyl acetate. Ethyl acetate is concentrated to a specific gravity of 1.10 g/cm³.
(d) Cooling the ethyl acetate phase to about 0° C., stirring and crystallizing for 12 hours, filtering and drying to obtain a sucralose-6-acetate crude product.

9.8 g of crude sucralose-6-acetate with a purity of 91.12% is obtained after the reaction. Compared with 32 g of initially-added sucralose-6-acetate, the obtained available sucralose-6-acetate is less, most sucralose-6-acetate is difficult to separate and purify due to the problems of ester soluble impurities and water soluble impurities, so that the sucralose-6-acetate is difficult to crystallize and finally is treated as waste residue. The traditional method causes great waste in the aspect of purification and utilization of the sucralose-6-acetate.

In conclusion, the application aims to treat the repeated mother solution of sucralose-6-ester by combining the oxidant and the alkaline hydrolysis agent so as to convert impurities such as sucralose diester and tetrachlorosucrose-6-ester in the mother solution into the target product sucralose-6-ester, thereby effectively removes the impurity content and greatly improves the yield of the target product; the whole treatment process is simple and smooth, the economic cost is low, and the practicability is strong; the purity of the obtained sucralose-6-ester is high, and the production of the sucralose can be directly carried out; the method can obviously reduce the impurity content of the wastewater, relieves the pressure of subsequent wastewater treatment, is environment-friendly, and has extremely high application and economic values.

In view of the foregoing, it is only a specific embodiment of the present application that other modifications and variations based on the above-described embodiments can be made by those skilled in the art in light of the above teachings. It should be understood by those skilled in the art that the foregoing detailed description is for the purpose of better explaining the present application, and the scope of the present application should be determined by the scope of the appended claims.

Moreover, those skilled in the art will appreciate that although some embodiments described herein include some features included in other embodiments, not others, combinations of features of different embodiments are meant to be within the scope of the application and form different embodiments. For example, in the following claims, any of the claimed embodiments may be used in any combination.

What is claimed is:

1. A method for purifying sucralose-6-ester, comprising an impurity conversion step: adding an oxidant into a mother solution to be purified, carrying out oxidation reaction under a first preset condition, then adding an alkaline hydrolysis agent into the mother solution, and carrying out alkaline hydrolysis reaction under a second preset condition to convert target impurities into sucralose-6-ester to obtain an oxidation alkaline hydrolysis reaction solution; wherein the mother solution to be purified is a repeated mother solution of sucralose-6-ester;
a pH adjusting step: adjusting the pH of the oxidation alkaline hydrolysis reaction solution to be neutral, keeping for a period of time, then heating and filtering to remove insoluble impurities, and separating liquid to obtain a first aqueous phase and a first organic phase;
an extraction step: extracting the first aqueous phase by using an extractant, combining a second organic phase obtained by extraction with the first organic phase to obtain a mother solution to be crystallized;
a crystallization step: concentrating the mother solution to be crystallized, and crystallizing under a third preset condition to obtain a crude sucralose-6-ester product and a first filtrate.

2. The method of claim 1, wherein prior to the impurity conversion step, the method further comprises a mother solution condensation step: condensing the mother solution to be purified to −10 to 30° C., preferably 0 to 10° C.

3. The method of claim 1, further comprising a refining step: dissolving the obtained crude sucralose-6-ester product in an extractant and recrystallizing under a fourth preset condition to obtain a refined sucralose-6-ester product.

4. The method of claim 3, wherein the step of dissolving the obtained crude sucralose-6-ester product in an extractant and recrystallizing under a fourth preset condition to obtain a refined sucralose-6-ester product comprises: dissolving the crude sucralose-6-ester product in an extractant with a mass ratio of 1-3:1, preferably 1.5-2:1 to the crude sucralose-6-ester product under the condition of heating, controlling the recrystallization temperature to be 5-15° C., preferably 0-5° C., and the recrystallization time to be 2-12 hours, preferably 6-10 hours, filtering and drying after crystallization is completed, to obtain a refined sucralose-6-ester product.

5. The method of claim 1, wherein the oxidant is hydrogen peroxide; the mass fraction of the hydrogen peroxide is 15-40%, preferably 25-35%; the volume of the hydrogen peroxide is 5-20%, preferably 10-15% of the volume of the mother solution to be purified.

6. The method according to claim 1, wherein the first preset condition is: the reaction temperature of the oxidation reaction is set to be −10 to 20° C., preferably 0 to 10° C.; the reaction time of the oxidation reaction is set to be 1-12 h, preferably 6-10 h.

7. The method of claim 1, wherein the alkaline hydrolysis agent is one or more of ammonia, triethylamine, diethylamine and dimethylamine; preferably, the alkaline hydrolysis agent is dimethylamine, wherein the concentration of dimethylamine is 10%-45 wt %, preferably 30%-40 wt %.

8. The method according to claim 1, wherein the second preset condition is: the reaction temperature of the alkaline hydrolysis reaction is set to be −10 to 30° C., preferably 0 to 10° C.; the reaction time of the alkaline hydrolysis reaction is set to be 3-12 h, preferably 6-10 h; the pH of the alkaline hydrolysis reaction is 7-12, preferably 9-10.

9. The method according to claim 1, wherein in the pH adjusting step, adjusting the pH of the oxidation alkaline hydrolysis reaction solution to be neutral, keeping for a period of time, then heating and filtering to remove insoluble impurities comprises: using an acidic substance to adjust the pH of the oxidation alkaline hydrolysis reaction solution to 6-8, preferably 7, keeping for 1-2 h, heating to 30-60° C., preferably 40-50° C., and then filtering out insoluble impurities; wherein the acidic substance is sulfuric acid, acetic acid or hydrochloric acid, preferably hydrochloric acid; the mass fraction of the acidic substance is 10-35%, preferably 25-30%.

10. The method of claim 1, wherein in the step of extracting, the extractant is ethyl acetate; the ratio of the volume of the extractant to the volume of the first aqueous phase is 0.5-4:1, preferably 1-2:1; the content of sucralose-6-ester in the second aqueous phase obtained after extraction is lower than 1 g/L.

11. The method according to claim 1, wherein in the crystallization step, the mother solution to be crystallized is concentrated by reduced pressure distillation, and the distillation temperature of the distillation is 30-80° C., preferably 35-55° C.; the distillation pressure is from −0.050 MPa to −0.99 MPa, preferably from −0.50 MPa to −0.8 MPa;
concentrating the mother solution to be crystallized to the density of 1-2 g/cm³, preferably 1.1-1.5 g/cm³.

12. The method according to claim 1, wherein in the crystallization step, the third preset condition is: the crystallization time is set to be 2-24 h, preferably 6-12 h; the crystallization temperature is set to be −10 to 15° C., preferably 0 to 5° C.
